(19) United States
(12) Patent Application Publication
(10) Pub. No.: US 2023/0356218 A1
(43) Pub. Date: Nov. 9, 2023

(54) CAVITY-SEPARATED MULTI-NANOPORE DEVICE AND METHOD PROVIDING PROTEIN SEQUENCING

(71) Applicant: The Trustees of Columbia University in the City of New York, New York, NY (US)

(72) Inventors: Jakob Buchheim, Ulm (DE); Kenneth Shepard, Ossining, NY (US)

(21) Appl. No.: 18/121,311

(22) Filed: Mar. 14, 2023

Related U.S. Application Data
(60) Provisional application No. 63/319,636, filed on Mar. 14, 2022.

(51) Int. Cl. B01L 3/00

(52) U.S. Cl. CPC B01L 3/502715; B01L 2300/0645; B01L 2300/0861

(57) ABSTRACT

An exemplary system and method can be provided, e.g., for detecting a molecular size and charge. The exemplary system can comprise a cavity, nanopores separated by the cavity, and electrolyte reservoirs. Each of the reservoirs can be provided on a side of a respective nanopore, and within the cavity. A plurality of such systems can be integrated in a surface of a complementary metal-oxide-semiconductor (CMOS) integrated circuit, which can comprise transimpedance amplifiers configured to measure a conductance through the nanopores. Further an exemplary device can be provided for protein sequencing, and can comprise a first compartment with a first electrode, a second compartment with a second electrode, and a channel between the first and second compartments. Each of the compartments can be fluidly coupled to the channel using a nanopore. A detector can also be provided which is configured to record at least one parameter in the channel by applying a voltage bias across the first and second electrodes so that charged molecules pass through the nanopore fluidly coupled to the first and second compartments.

### CROSS REFERENCE TO RELATED APPLICATION(S)

This application relates to and claims the benefit of priority from U.S. Provisional Pat. Application No. 63/319,636, filed Mar. 14, 2022, the entire disclosure of which is incorporated herein by reference in its entirety.

### STATEMENT REGARDING FEDERALLY SPONSORED RESEARCH

This invention was made with government support under grant nos. NS099717 and HG009189 awarded by the National Institutes of Health. The government has certain rights in the invention.

### FIELD OF THE DISCLOSURE

The present disclosure relates generally to protein sequencing, and more specifically, to exemplary embodiments of exemplary cavity-separated multi-nanopore (“CSMP”) device and method which can facilitate protein sequencing.

### BACKGROUND INFORMATION

Referring to Edman degradation and mass spectroscopy, some methods have even tried to combine these techniques. [See, e.g., Refs. 1-3]. In Edman degradation [see, e.g., Ref. 4], a series of chemical reactions are used to label, cleave, and identify amino acids as they are cleaved from the N- to the C-terminus one at a time. The ability to control these reactions can limit Edman degradation to purified peptides shorter than 50 amino acids. The other challenge can be that each degradation cycle can take up to 45 minutes. [See, e.g., Ref. 5].

In mass-spectroscopy(“MS”)-based protein identification [see, e.g., Ref. 6], proteins are fragmented into peptides which are separated with chromatography, ionized, and captured by the mass spectrometer. The peaks of each peptide fragment ion is then used for identification, which is performed in one of two ways, either a database search or de novo sequencing. In the data-base-search approach, theoretical spectra are created from a protein database and the closest match is identified. Many methods have been developed to do this match, including SEQUEST [see, e.g., Ref. 7], X/TANDEM [see, e.g., Ref. 8], OMASSA [see, e.g., Ref. 9], and MASCOT [see, e.g., Ref. 10], but this only likely works if the protein of interest is known and in the database. By contrast, in de novo sequencing, proteins are identified directly from the spectrum peaks. Identification is made by finding the longest possible peptide sequence that matches the experimental spectrum. [See, e.g., Refs. 11-13].

These methods are effectively single-channel systems, limited in throughput, dynamic range, and sensitivity. Genomic analysis manages the detection of low concentrations with polymerase chain reaction (PCR) amplification, which is not available for proteins. Within a mammalian cell, protein concentrations span approximately eight orders of magnitude; in human blood, this increases to eleven orders of magnitude. This can be important because in the complex environments, even the least expressed proteins may carry great significance in cellular signaling, gene regulation, and disease onset. State-of-the-art MS instrumentation offers a dynamic range on the order of only 104 or 105. Detection limits for MS are also only on the order of 0.1 to 10 femtomole. For proteins expressed at the level of 1000 proteins per cell (on the order of an attomole), millions of cells would be required to produce enough protein for analysis

Because of these limitations in conventional techniques, there is growing interest in next-generation protein sequencing approaches that would enable single-cell analysis. One of the more successful approaches to single-molecule protein identification is that based on fluorescent fingerprinting. [See, e.g., Refs. 14 and 15]. In such case, two amino acids are labelled fluorescently, cysteine (C) and lysine (K). These are sequential detected through the action of the unfoldase ClpX, which is immobilized on a glass surface and unfolds the labelled polypeptide and translocate it. The enzyme itself is labelled with a donor fluorophore with acceptor fluorophores on the C and K amino acids. The associated fluorescence resonance energy transfer (FRET) signal is detected with time-internal reflection (TIRF) microscopy. Even labelling only two amino acids, the technique is able to detect a significant number of proteins (>70-80%) with database searches but with error rates on the order of 20-30%. [See, e.g., Ref. 14].

The limitations in fluorescent approaches have led to considerable interest in translating approaches for nanopore-based DNA sequencing to proteins. The most successful approaches to nanopore-based DNA sequencing rely on ratcheting the DNA through the pore by the action of an enzyme stepping motor, usually in the form of a polymerase [see, e.g., Ref. 16]. This allow translocation rates and molecular entropy to be controlled and individual nucleotides to be stepped one-by-one through the pore for analysis based on ion conduction. Efforts to develop similar systems with proteins have focused on the same unfoldases used in molecular fingerprinting, such a ClpX [see, e.g., Ref. 17], which can unfold and pull proteins through pores with ATP hydrolysis. [See, e.g., Refs. 18 and 19]. The motor in these cases can been positioned at either the exit or entrance to the pore. These approaches are challenging to the difficulty in distinguishing 20 amino acids by ion current effects along.

An alternative to this “thread-and-read” approach is one that has been deemed “chop-and-drop.” (See, e.g., Ref. 20]. In this case, a peptidase cleaves the protein at the nanopore entrance and the protein fragments are analyzed as they are sequentially cleaved. In many ways, this is similar to exonuclease nanopore sequencing approaches, which require that nucleotides be cleaved off close enough to the pore to guarantee capture and that transit of the nucleotide must be slow enough to be able to measure the ionic signal. [See, e.g., Refs. 21-23]. Chop-and-drop approaches have many features in common with MS approaches in requiring proteins to be cleaved and in employing the nanopore as a kind of mass spectrometer. The problem is that relying on ion current blockage alone as the only sensing modality does not provide adequate information for fine mass determination.

Thus, it may be beneficial to provide exemplary cavity-separated multi-nanopore (CSMP) device and method which can facilitate protein sequencing, which can overcome at least some of the deficiencies described herein above.

### SUMMARY OF EXEMPLARY EMBODIMENTS

Accordingly, such exemplary device and method is described herein. For example, according to an exemplary embodiment of the present disclosure, a chop-and-drop sequencing approach can be provided based on a cavity-separated system of multiple solid-state nanopores. This can facilitate the nanopore to not only detect ionic currents, but also the time it takes the fragmented peptide to traverse from one pore to the other, thereby, among other things, providing further information on the fragment size.

To that end, according to an exemplary embodiment of the present disclosure, an exemplary cavity-separated multi nanopore (CSMP) device can be provided for protein sequencing. The CSMP separates to fluidic compartments containing electrolytic solution. For example, electrodes in each compartment facilitate an application of a voltage bias across the CSMP, and therefore drive charge carriers and other charged molecules through CSMP. The resulting ionic current can be recorded over time with a high bandwidth low-noise current amplifier. While translocating through the constrictions of the CSMP device, a protein/polypeptide/peptide/amino acid can cause a specific pattern of ionic current modulation in current amplitude and duration which facilitates an identification of the translocating molecule. Protein sequencing can be facilitated by an identification of individual peptides or amino acids stemming from the protein one after the other, by the specific current and dwell time of ionic current measurement recorded when the peptides translocate through the CSMP device.

According to another exemplary embodiment of the present disclosure, the CSMP system can include and/or be a nanopore device and the fabrication of the very same, which can incorporate at least two membranes both with, e.g., less than 10 nm thickness and pores smaller than or equal to, e.g., 5 nm separated by a cavity of, e.g., larger than 50 nm and nanoscale diameter.

In a further exemplary embodiment of the present disclosure, a cavity wetting method can be provided to overcome a capillary pressure occurring when filling nanoscale hollow cavities by including a sacrificial material inside the cavity which dissolves upon an initial device wetting.

According to yet another exemplary embodiment of the present disclosure, it is possible to provide an exemplary configuration in which a complementary metal-oxide-semiconductor (CMOS) ionic current amplifier can be combined with the above described exemplary nanopore sensing membrane integrated directly and/or in a chip-to-chip manner to facilitate a low input capacitance, e.g., C_{I}< about 1.0 pF, low noise smaller than vₙ = about 1.8 nV/Hz^{⅟} and/or high bandwidth greater or equal to about 10 MHz sampling.

In still another exemplary embodiment of the present disclosure, it is possible to measure and record signals of multiple CSMP devices, e.g., at approximately the same time and/or in a substantially parallel manner to facilitate multiple protein sequencing at once.

According to a still another exemplary embodiment of the present disclosure, it is possible to provide a surface immobilized protein cleavage method which can facilitate a site specific degradation of proteins and product release in the vicinity of the CSMP entrance. In a still further exemplary embodiment of the present disclosure, it is possible facilitate trapping of a protein in the vicinity of the pore entrance by a diffusive, electrophoretic or flow field trap for example, but not limited to, a nanowell confinement to facilitate, e.g., a direct / online enzymatic degradation of proteins and polypeptides to small peptides before there translocation. In addition, according to a yet further exemplary embodiment of the present disclosure, it is possible to provide an exemplary method to reduce protein sequencing errors by using one or more site specific protein cleavage methods and the identification of their cleavage products providing complementary information on the protein sequence.

According to yet another exemplary embodiment of the present disclosure, an exemplary system can be provided, e.g., for detecting a molecular size and a molecular charge. Such exemplary system can comprise a cavity, a plurality of nanopores separated by the cavity, and a plurality of electrolyte reservoirs. Each of the reservoirs can be provided on a side of a respective one of the nanopores, and within the cavity.

For example, at least one of the nanopores can be fabricated with two-dimensional materials and/or in a silicon nitride membrane. The particular ones of the reservoirs provided on the sides nanopores can be denoted as a cis chamber and a trans chamber, respectively. Further, a nanowell and/or a single protease can be positioned at one of entrances of at least one of the nanopores. A plurality of proteases can be positioned in a well.

According to a still another exemplary embodiment of the present disclosure, a plurality of such systems can be integrated in a surface of a CMOS integrated circuit, which can comprise a plurality of transimpedance amplifiers configured to measure a conductance through the nanopores.

Further, an exemplary device can be provided for protein sequencing. Such exemplary device can comprise a first compartment which includes a first electrode, a second compartment which includes a second electrode, and a channel provided between the first compartment and the second compartment. Each of the first compartment and the second compartment can be fluidly coupled to the channel using a nanopore. A detector can also be provided which is configured to record at least one parameter in the channel by applying a voltage bias across the first electrode and the second electrode so that charged molecules pass through the nanopore fluidly coupled to the first compartment and the second compartment.

For example, the parameter(s) can be a current, a travel time of the charged molecules within the channel, a mobility of the charged molecules within the channel, and/or a charge volume. An integrated amplifier can be provided below the channel.

Further, according to another exemplary embodiment of the present disclosure, a method can be provided for fabricating a device for protein sequencing. Using this exemplary method, it is possible to create a channel between a first compartment and a second compartment of a device, whereas each of the first compartment and the second compartment is fluidly coupled to the channel via a nanopore. Then, it is possible to provide a sacrificial layer within the channel, dissolve the sacrificial layer, and fabricate the device using the channel once the sacrificial layer is dissolved.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a schematic diagram of an exemplary cavity-separated multi nanopore CSMP device in accordance with the exemplary embodiment of the present disclosure;

FIG. 2 is an illustration of an exemplary dataset for recorded I(t) from a translocation of protein through cavity-separated double nanopore indicating exemplary characteristic measurement values ΔI_{P1}, τ_{P1}, ΔI_{P2}, τ_{P2}. ΔI_{cavity} and τ in accordance with the exemplary embodiment of the present disclosure;

FIG. 3 is an illustration of exemplary fabrication steps for manufacturing the exemplary CSMP device in accordance with the exemplary embodiment of the present disclosure;

FIG. 4A is a schematic side cutaway view of nanowell and immobilized protease, with a cavity-separated dual nanopore and the nanowell on cis side in accordance with an exemplary embodiment of the present disclosure;

FIG. 4B is a tilted view of nanowell and immobilized protease, with tilted view of an immobilized enzyme in the bottom of the nanowell in accordance with an exemplary embodiment of the present disclosure; and

FIG. 5 is a schematic diagram of an exemplary CMOS integrate chip current amplifier stage for a single channel CSMP ionic current recording in accordance with an exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIEMNTS

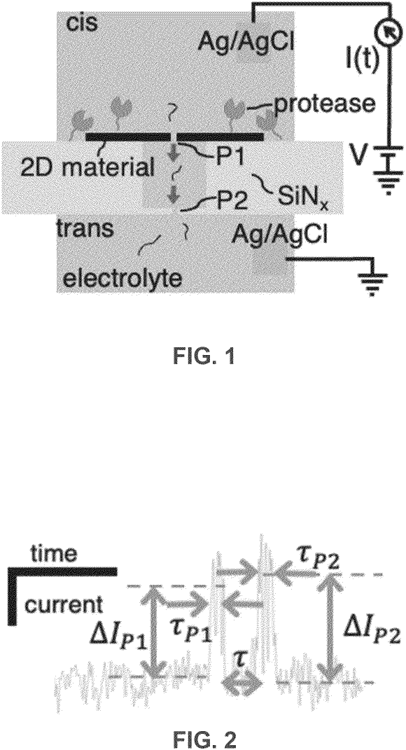

According to an exemplary system, method and computer-accessible medium, according to exemplary embodiments of the present disclosure, the exemplary design and fabrication of cavity-separated multiple nanopore device can be provided and its usage for protein sequencing. The exemplary nanopore device can be fabricated or otherwise manufactured on top of, e.g., <100> Si chip with greater than about 2 µm of silicon oxide (SiO₂) and greater than about 50 nm thick silicon nitride (SiNₓ) or other low capacitance carriers which can facilitate a through substrate vias, and which can support greater than about 50 nm thick free-standing membranes, as shown in FIG. 1. Indeed, FIG. 1 illustrates a schematic diagram of an exemplary cavity-separated multi nanopore CSMP device 100 in accordance with the exemplary embodiment of the present disclosure. For an exemplary dual nanopore system 100, a 2d material membrane 110 can enclose a cavity 120 fabricated inside a freestanding low stress thin film 130 made form SiNx or similar material. In both exemplary membrane layers 110, a single pore P1 and P2 having smaller or equal to about 5 nm diameter can be drilled. The entire membrane 110 can separate to otherwise sealed fluidic compartments, and/or can be immersed in an electrolyte solution. By applying a voltage bias across the device, a ionic current can thus be caused to flow through the motion of ions and other molecules passing the membrane.

The resulting ionic current I(t) can be recorded over time with high bandwidth, as shown in FIG. 2. Indeed, FIG. 2 illustrates an exemplary dataset for recorded I(t) from a translocation of protein through cavity-separated double nanopore indicating exemplary characteristic measurement values ΔI_{P1}, τ_{P1}, ΔI_{P2}, τ_{P2.} ΔI_{cavity} and τ in accordance with the exemplary embodiment of the present disclosure.

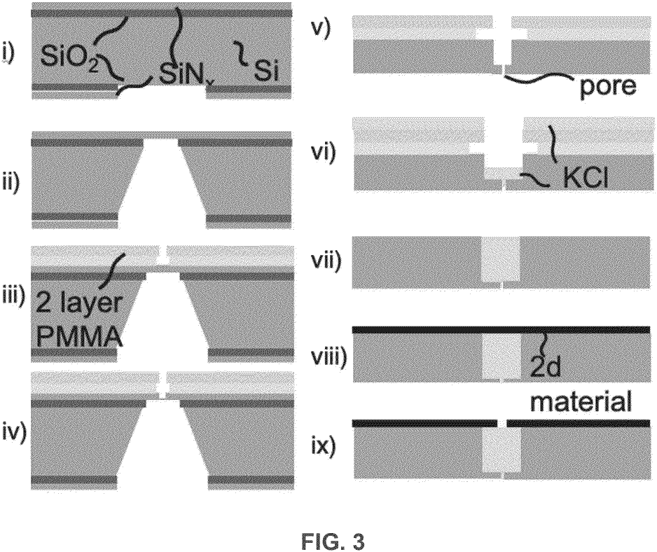

The exemplary devices according to various exemplary embodiments of the present disclosure can be fabricated or otherwise manufactured on, e.g., the exemplary described silicon support frame, as shown in FIG. 3, e.g., on a <100> Si Substrate 310 coated with less than about 2 um SiO₂ 320 and with less than about 50 nm low stress SiNx 330 on both sides. For example, section (i) of FIG. 3 illustrates a hard mask generation for through wafer anisotropic etching of SiNx using TMAH or KOH.

This can be done by, e.g., by creating or otherwise providing a hard mask for anisotropical etching of the silicon in KOH or TMAH solution until the wafer is etched all the way through and the SiNₓ membrane is released, e.g., resulting free standing low stress SiNx membrane after BOE etched buried oxide layer (see FIG. 3, panel ii).

Spin coating 2 layer resist with undercut can be provided to facilitate reliable lift off after material deposition. For example, a two-layer e-beam resist can be (e.g., spin-) coated and/or patterned by, e.g., high resolution lithography in the free standing SiNₓ membrane area to define the cavity lateral dimensions, e.g., with undercut to facilitate a reliable lift off after material deposition, which can subsequently acts as a liftoff photoresist layer for the KCl deposition (see FIG. 3, panel iii). Poly(methyl methacrylate) (or “PMMA”) can be patterned by electron beam lithography or similar high resolution technique.

The cavity 350 can be formed (e.g., inside low stress freestanding membrane) by thinning down locally the free standing SiNx membrane in the desired area by focused ion beam or by using wet etching to a remaining SiNx membrane thickness of, e.g., about 5 nm (see FIG. 3, panel iv). For example, the first nanopore can be created by dielectric breakdown drilling, focused ion beam patterning [see, e.g., Ref. 24] or TEM patterning [see, e.g., Ref. 25] into the remaining approx. 5 nm SiNₓ membrane following well-established protocols [see, e.g., Refs. 26 and 27]. FIG. 3, panel v illustrates an exemplary pore 360 formed inside the exemplary cavity 360 defining a membrane layer 370. To stabilize, e.g., the first nanopore for the high saline aquouse solutions, an additional ALD coating with HfO₂ or TiO₂ can be employed. The first pore can be, e.g., fully characterized with current-voltage recordings at different salt concentrations.

To facilitate wetting of the later enclosed nanoscale cavity, a sacrificial material 380 for example but not limited to KCl is evaporated onto the device, filling up the cavity from the bottom, e.g., by depositing the sacrificial material (e.g., KCl) into the cavity 360 with, e.g., pattern defining liftoff mask (see FIG. 3, panel vi). The sacrificial material layer (e.g., KCl) can be lifted off in a non-polar anhydrous or with insoluble material solvent, e.g., like chloroform for which PMMA has an excellent solubility (see FIG. 3, panel vii).

The cavity 360 can be closed with the second membrane using well established dry transfer technique for 2D material 390, e.g., by transferring 2D material on top of cavity to seal it, with dry or wet transfer techniques being usable (see FIG. 3, panel viii), as also described in a publication [see, e.g., Ref. 28]. h-BN, MoS₂ or graphene have been yielded stable nanometer-sized solid-state nanopores [see, e.g., Refs. 29-31]. Indeed, FIG. 3, panel ix shows an exemplary formation of the second exemplary pore 395 in the second exemplary material.

Any polymer residuals from the transfer process can be cleanly removed by annealing the sample in atmospheric pressures in hydrogen-containing gas mixtures. The second pore can also be created by dielectric breakdown drilling or Focused Ion Beam milling. During the dielectric breakdown process, as long as the second membrane is still intact, the electric field can mainly drop across this membrane. Once the current flows after the creation of the second pore, the first pore can then experience a potential drop. For example, the dielectric breakdown of pores in series can create pores with similar resistances and pore sizes for comparable membrane thicknesses. According to an exemplary non-limiting exemplary embodiment of the present disclosure, such fabricated cavity-separated dual nanopore devices can have exemplary pore sizes, e.g., between about 0.5 and 5 nm diameter, exemplary membrane thickness of, e.g., between about 0.6 and 5 nm, exemplary cavity sizes, e.g., between about 50 to 500 nm length and about 20 to 300 nm diameters. The overall exemplary capacitance can be, e.g., below 1pC inside 1 M KCl solution.

According to another alternative and/or additional exemplary embodiment of the present disclosure, an exemplary direct integration or an exemplary chip-to-chip integration of a CMOS ionic current amplifier which supports bandwidth of great than or equal to, e.g., about 10 MHz, with a low input referred noise of vₙ being less than or equal to, e.g., about 1.8 nV/Hz^{½} can be provided.

The exemplary direct integration provided a beneficial (and likely best) noise performance because the distance between the cis electrode and the amplifier input is reduced to, e.g., less than about 100 um, thereby reducing the propensity for electronic interference and contributing minimal interconnection capacitance. Both the cis and trans chambers can be coupled to the electronics with Ag/AgCl electrodes. For example, the cis electrode, which connects to the input of the amplifier, can be fabricated or otherwise provided onto the surface of the amplifier chip, which can be accomplished by, e.g., electroplating Ag onto existing metal pads on the CMOS chip, followed by chemical chlorination. The surface electrode can become exhausted after several hours, although it can be regenerated with, e.g., re-electroplating. The trans electrode can be externally connected, since the capacitance at this electrode does not affect the noise floor.

For example, to achieve the beneficial and/or required temporal resolution in, e.g., the 0.1 us scale and low noise current at frequencies beyond about 2 MHz, the input revered voltage noise can be decreased and additional noise source in the active feedback resistors can be significantly reduced or eliminated. The exemplary input-referred voltage noise floor vₙ of CMP3 can be reduced down to, e.g., about 1.8

$$n V / \sqrt{H z} .$$

As vₙ is inversely proportional to the transconductance of a transistor (gₘ), the noise floor for transistors already biased in the deep subthreshold regime can be improved by passing more current through them which can, e.g., consequently increase gₘ and reduce vₙ.

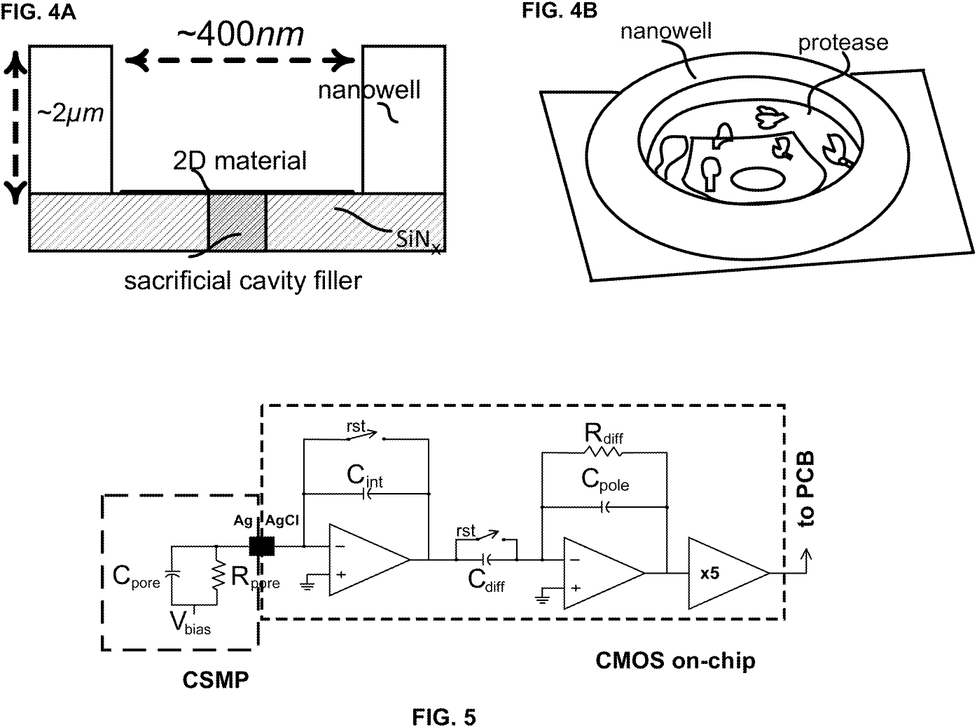

In general, CMOS technology does not facilitate for the use of linear, large-valued resistors. To avoid noise of reversed biased diodes in the active resistor feedback, an alternative exemplary circuit based on an integrator followed by a differentiator can be provided, as shown in FIG. 5. In particular, FIG. 5 illustrates a schematic diagram of an exemplary CMOS integrate chip current amplifier stage 510 for a single channel CSMP ionic current recording according to an exemplary embodiment of the present disclosure. Using such exemplary circuit shown in FIG. 5, the low-pass transimpedance gain response can be retained while removing the feedback resistance to the input. Input current causes charge to accumulate on the feedback integration capacitor which the opamp output adjusts to maintain a zero differential input voltage. A parallel switch 520 (as shown in FIG. 5) can be used to discharge the integration capacitor, thereby, e.g., preventing the opamp output from saturating. The exemplary two-phase switching scheme can be used for charge-neutral discharging during reset.

In particular, referring to FIG. 5, e.g., a discharged copy of Cint and C_{diff} can be switched “in” while the resetting capacitor has its terminals shorted, facilitating continuous measurements and minimized reset transients. For example, a gain of 1 or 10 can be provided by the C_{diff}/Cᵢₙₜ ratio. The differentiator stage can restore the transimpedance gain to a low-pass response with a cutoff frequency of, e.g., about 1 MHz with a DC gain of about 100 MHz. The final output buffer stage can provide, e.g., a 5V/V gain before filtering. The CMOS amplifier 510 can comprise multiple independent channels to facilitate parallel recording of more than one CSMP devices 530 at the same time.

According to yet another alternative and/or additional exemplary embodiment of the present disclosure, the exemplary devices and methods can utilize low distortion data filtering based on wavelets to retain maximum precision in dwell time measurements. For example, since high precision dwell time measurement can be needed for peptide identification, exemplary low distortion data filtering should be employed. Conventionally, low-pass Bessel filters are employed to reduce the noise level of the measured current trace. However, the square pulse-like shape of current recordings can imply that the frequency content of such signals is not well-localized. As a result, filters designed in the frequency domain generally suppress the noise and signal content. Wavelet transform-based denoising offers an alternative [see, e.g., Refs. 32 and 33], as it is used in image denoising where sharp signal edges are common and should be preserved [see, e.g., Ref. 34]. Instead, an exemplary wavelet-transform based denoising procedure(s) [see, e.g., Ref. 35] can be utilized in accordance with the exemplary embodiment of the present disclosure to significantly reduce or suppress noise without sacrificing on edge sharpness.

According to an exemplary embodiment of the present disclosure, it is possible to integrate a diffusive trap based on a nanowell confinement, and/or provide an electrophoretic transport or a flow field to trap proteins for longer times at the vicinity of the CSMP device. For example, a nanowell 410 (as shown in FIGS. 4A and 4B) can functions as diffusive trap for large proteins increasing their residence time at the pore entrance. For example, FIG. 4A illustrates a schematic side cutaway view of nanowell 410 and immobilized protease, with a cavity-separated dual nanopore and the nanowell 410 on cis side in accordance with an exemplary embodiment of the present disclosure, and FIG. 4B shows a tilted view of nanowell 410 and immobilized protease 420, with tilted view of an immobilized enzyme 440 in the bottom of the nanowell in accordance with an exemplary embodiment of the present disclosure. For example, the bottom of the nanowell 410 around the pore entrance 430 can be grafted with immobilized protease 420 which can support protein degradation. The cleaved peptides can be trapped by the field gradient at the entrance of the CSMP and translocated through the nanopore device. Peptide identification can be facilitated by mapping the specific ionic current pattern, e.g., ΔI_{P1}, τ_{P1}, ΔI_{P2}, τ_{P2}. ΔI_{cavity} and τ (see FIG. 2) to a peptide database identifying the molecule uniquely. According to an exemplary embodiment of the present disclosure, it is possible to parallelized readouts and incorporate different protease at different CSMP devices which can yield complementary readout signals for the cleaved protein.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification and drawings, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### EXEMPLARY REFERENCES

The following reference is hereby incorporated by references in their entireties:

1. Miyashita M, Presley JM, Buchholz BA, Lam KS, Lee YM, Vogel JS, Hammock BD. Attomole level protein sequencing by Edman degradation coupled with accelerator mass spectrometry. Proceedings of the National Academy of Sciences. 2001;98(8):4403-8.

2. SHIMONISHI Y, HONG YM, KITAGISHI T, MATSUO T, MATSUDA H, KATAKUSE I. Sequencing of peptide mixtures by Edman degradation and field-desorption mass spectrometry. European journal of biochemistry. 1980;112(2):251-64.

3. Bradley CV, Williams DH, Hanley MR. Peptide sequencing using the combination of Edman degradation, carboxypeptidase digestion and fast atom bombardment mass spectrometry. Biochem Biophys Res Comm. 1982;104(4):1223-30.

4. Edman P, Högfeldt E, Sillén LG, Kinell P-O. Method for determination of the amino acid sequence in peptides. Acta chem scand. 1950;4(7):283-93.

5. Restrepo-Pérez L, Joo C, Dekker C. Paving the way to single-molecule protein sequencing. Nature nanotechnology. 2018;13(9):786-96.

6. Yates III JR. A century of mass spectrometry: from atoms to proteomes. nature methods. 2011;8(8):633-7.

7. Eng JK, McCormack AL, Yates JR. An approach to correlate tandem mass spectral data of peptides with amino acid sequences in a protein database. Journal of the american society for mass spectrometry. 1994;5(11):976-89.

8. Craig R, Beavis RC. TANDEM: matching proteins with tandem mass spectra. Bioinformatics. 2004;20(9):1466-7.

9. Geer LY, Markey SP, Kowalak JA, Wagner L, Xu M, Maynard DM, Yang X, Shi W, Bryant SH. Open mass spectrometry search algorithm. Journal of proteome research. 2004;3(5):958-64.

10. Perkins DN, Pappin DJ, Creasy DM, Cottrell JS. Probability-based protein identification by searching sequence databases using mass spectrometry data. ELECTROPHORESIS: An International Journal. 1999;20(18):3551-67.

11. Dančík V, Addona TA, Clauser KR, Vath JE, Pevzner PA. De novo peptide sequencing via tandem mass spectrometry. Journal of computational biology. 1999;6(3-4):327-42.

12. Johnson RS, Taylor JA. Searching sequence databases via de novo peptide sequencing by tandem mass spectrometry. Molecular biotechnology. 2002;22(3):301-15.

13. Frank A, Pevzner P. PepNovo: de novo peptide sequencing via probabilistic network modeling. Analytical chemistry. 2005;77(4):964-73.

14. Yao Y, Docter M, Van Ginkel J, de Ridder D, Joo C. Single-molecule protein sequencing through fingerprinting: computational assessment. Physical biology. 2015;12(5):055003.

15. Van Ginkel J, Filius M, Szczepaniak M, Tulinski P, Meyer AS, Joo C. Single-molecule peptide fingerprinting. Proceedings of the National Academy of Sciences. 2018;115(13):3338-43.

16. Manrao EA, Derrington IM, Laszlo AH, Langford KW, Hopper MK, Gillgren N, Pavlenok M, Niederweis M, Gundlach JH. Reading DNA at single-nucleotide resolution with a mutant MspA nanopore and phi29 DNA polymerase. Nature Biotechnol. 2012. doi: 10.1038/nbt.2171.

17. Olivares AO, Baker TA, Sauer RT. Mechanical protein unfolding and degradation. Annu Rev Physiol. 2018;80:413-29.

18. Nivala J, Marks DB, Akeson M. Unfoldase-mediated protein translocation through an α-hemolysin nanopore. Nature Biotechnol. 2013;31(3):247-50.

19. Nivala J, Mulroney L, Li G, Schreiber J, Akeson M. Discrimination among protein variants using an unfoldase-coupled nanopore. ACS nano. 2014;8(12):12365-75.

20. Zhang S, Huang G, Versloot R, Herwig BM, de Souza PCT, Marrink S-J, Maglia G. Bottom-up fabrication of a multi-component nanopore sensor that unfolds, processes and recognizes single proteins. bioRxiv. 2020.

21. Clarke J, Wu H-C, Jayasinghe L, Patel A, Reid S, Bayley H. Continuous base identification for single-molecule nanopore DNA sequencing. Nature nanotechnology. 2009;4(4):265-70.

22. Reiner JE, Balijepalli A, Robertson JW, Drown BS, Burden DL, Kasianowicz JJ. The effects of diffusion on an exonuclease/nanopore-based DNA sequencing engine. The Journal of chemical physics. 2012;137(21):214903.

23. Fuller CW, Kumar S, Porel M, Chien M, Bibillo A, Stranges PB, Dorwart M, Tao C, Li Z, Guo W. Real-time single-molecule electronic DNA sequencing by synthesis using polymer-tagged nucleotides on a nanopore array. Proceedings of the National Academy of Sciences. 2016;113(19):5233-8.

24. Buchheim J, Wyss RM, Shorubalko I, Park HG. Understanding the interaction between energetic ions and freestanding graphene towards practical 2D perforation. Nanoscale. 2016;8(15):8345-54.

25. Storm AJ, Chen JH, Ling XS, Zandbergen HW, Dekker C. Fabrication of solid-state nanopores with single-nanometre precision. Nature Materials. 2003;2(8):537-40. doi: 10.1038/nmat941.

26. Briggs K, Kwok H, Tabard-Cossa V. Automated Fabrication of 2-nm Solid-State Nanopores for Nucleic Acid Analysis. Small. 2014;10(10):2077-86.

27. Waugh M, Briggs K, Gunn D, Gibeault M, King S, Ingram Q, Jimenez AM, Berryman S, Lomovtsev D, Andrzejewski L. Solid-state nanopore fabrication by automated controlled breakdown. Nat Protocols. 2020;15(1):122-43.

28. Dean CR, Young AF, Meric I, Lee C, Wang L, Sorgenfrei S, Watanabe K, Taniguchi T, Kim P, Shepard KL. Boron nitride substrates for high-quality graphene electronics. Nature nanotechnology. 2010;5(10):722.

29. Zhou J, Thompson B, Hess RF. A new form of rapid binocular plasticity in adult with amblyopia. Sci Rep. 2013;3(1):1-5.

30. Thiruraman JP, Masih Das P, Drndic M. Stochastic Ionic Transport in Single Atomic Zero-Dimensional Pores. ACS nano. 2020;14(9):11831-45.

31. Merchant CA, Healy K, Wanunu M, Ray V, Peterman N, Bartel J, Fischbein MD, Venta K, Luo Z, Johnson ATC, Drndić M. DNA Translocation through Graphene Nanopores. Nano Letters. 2010;10(8):2915-21. doi: 10.1021/n1101046t.

32. Donoho DL, Johnstone IM. Ideal spatial adpatation by wavelet shrinkage. Biometrika. 1994;81:425-55.

33. Donoho DL, Johnstone IM. Adapting to unknown smoothness via wavelet shrinkage. Journal of the American Statistical Association. 1995;90:1200-24. doi: 10.1080/01621459.1995.10476626.

34. Chang SG, Yu B, Vetterli M. Adaptive wavelet thresholding for image denoising and compression. IEEE Transactions on Image Processing. 2000;9:1532-46. doi: 10.1109/83.862633. PubMed PMID: 18262991.

35. Shekar S, Chien C-C, Hartel A, Ong P, Clarke OB, Marks A, Drndic M, Shepard KL. Wavelet Denoising of High-Bandwidth Nanopore and Ion-Channel Signals. Nano letters. 2019;19(2): 1090-7.

What is claimed is:

1. A system for detecting a molecular size and a molecular charge, comprising:
a cavity;
a plurality of nanopores separated by the cavity; and
a plurality of electrolyte reservoirs, each of the reservoirs being provided (i) on a side of a respective one of the nanopores, and (ii) within the cavity.

2. The system of claim 1, wherein at least one of the nanopores is fabricated with two-dimensional materials.

3. The system of claim 1, wherein at least one of the nanopores is fabricated in a silicon nitride membrane.

4. The system of claim 1, wherein the particular ones of the reservoirs provided on the sides nanopores are denoted as a cis chamber and a trans chamber, respectively.

5. The system of claim 1, further comprising a nanowell positioned at one of entrances of at least one of the nanopores.

6. The system of claim 1, further comprising a single protease nanopores.

7. The system of claim 1, further comprising a plurality of proteases positioned in a well.

8. A complementary metal-oxide-semiconductor (CMOS) integrated circuit, comprising:
a plurality of systems for detecting a molecular size and a molecular charge, at least one of the systems comprising:
a cavity,
a plurality of nanopores separated by the cavity, and
a plurality of electrolyte reservoirs, each of the reservoirs being provided (i) on a side of a respective one of the nanopores, and (ii) within the cavity,
wherein the systems are integrated onto a surface of the circuit; and
a plurality of transimpedance amplifiers configured to measure a conductance through the nanopores.

9. The CMOS integrated circuit of claim 8, wherein at least one of the nanopores is fabricated with two-dimensional materials.

10. The CMOS integrated circuit of claim 8, wherein at least one of the nanopores is fabricated in a silicon nitride membrane.

11. The CMOS integrated circuit of claim 8, wherein the particular ones of the reservoirs provided on the sides nanopores are denoted as a cis chamber and a trans chamber, respectively.

12. The CMOS integrated circuit of claim 8, wherein the at least one of the systems comprises a nanowell positioned at one of entrances of at least one of the nanopores.

13. The CMOS integrated circuit of claim 8, wherein the at least one of the systems comprises a single protease nanopores.

14. The CMOS integrated circuit of claim 8, wherein the at least one of the systems comprises a plurality of proteases positioned in a well.

15. A device for protein sequencing, comprising:
a first compartment which includes a first electrode;
a second compartment which includes a second electrode;
a channel provided between the first compartment and the second compartment, wherein each of the first compartment and the second compartment is fluidly coupled to the channel using a nanopore; and
a detector configured to record at least one parameter in the channel by applying a voltage bias across the first electrode and the second electrode so that charged molecules pass through the nanopore fluidly coupled to the first compartment and the second compartment.

16. The device of claim 15, wherein the at least one parameter is at least one of a current, a travel time of the charged molecules within the channel, a mobility of the charged molecules within the channel, or a charge volume.

17. The device of claim 16, further comprising an integrated amplifier provided below the channel.

18. A method for fabricating a device for protein sequencing, the method comprising:
creating a channel between a first compartment and a second compartment of a device, wherein each of the first compartment and the second compartment is fluidly coupled to the channel via a nanopore;
providing a sacrificial layer within the channel;
dissolving the sacrificial layer; and
fabricating the device using the channel once the sacrificial layer is dissolved.
